(19) United States
(12) Patent Application Publication
(10) Pub. No.: US 2026/0061006 A1
(43) Pub. Date: Mar. 5, 2026

(54) ALLOGENEIC MESENCHYMAL STEM CELLS FOR THE TREATMENT OF RETINAL DISORDERS ASSOCIATED WITH THE PRESENCE OF DRUSEN

(71) Applicants: Anthony Oliva, West Miami, FL (US); Douglas Oliver, Nashville, TN (US); David Almeida, Erie, PA (US)

(72) Inventors: Anthony Oliva, West Miami, FL (US); Douglas Oliver, Nashville, TN (US); David Almeida, Erie, PA (US)

(21) Appl. No.: 18/963,295

(22) Filed: Nov. 27, 2024

Related U.S. Application Data
(60) Provisional application No. 63/689,516, filed on Aug. 30, 2024.

(51) Int. Cl. A61K 35/28; A61K 9/00; A61K 47/20; A61P 27/02

(52) U.S. Cl. CPC A61K 35/28; A61K 9/0019; A61K 9/0048; A61K 47/20; A61P 27/02

(57) ABSTRACT

The present disclosure provides for one or more embodiments for treating retinal degenerative disorders in a subject by administering to the subject an effective amount of allogeneic mesenchymal stem cells to the subject's suprachoroidal area. Allogeneic mesenchymal stem cells, when used in the disclosed treatment regimen, may exhibit anti-inflammatory, immunomodulatory, anti-fibrotic, pro-vascular, and/or pro-regenerative processes. Thus, allogeneic mesenchymal stem cells may be understood to have the potential to target and treat multiple pathological targets implicated in retinal degenerative disorders, including retinal disorders characterized by drusen accumulation, some of which have inherited forms.

### CROSS-REFERENCES TO RELATED APPLICATIONS

This application claims benefit of the following patent application(s) which is/are hereby incorporated by reference: U.S. Provisional Patent App. No. 63/689,516 filed on Aug. 30, 2024.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### STATEMENT REGARDING FEDERALLY SPONSORED RESEARCH OR DEVELOPMENT

Not Applicable

### REFERENCE TO SEQUENCE LISTING OR COMPUTER PROGRAM LISTING APPENDIX

Not Applicable

### BACKGROUND

The present invention relates generally to compositions and treatments for retinal disorders associated with the presence of drusen.

Drusen-related retinal disorders are a class of retinal degenerative diseases characterized by pathological accumulation of extracellular deposits in the retina, called drusen, that are generally small deposits being yellow or white in appearance, and composed of proteins, lipids, and cellular debris. These retinal disorders are often accompanied by chronic inflammation of the retina, impairment of retina pigment epithelial cell function, and geographic atrophy. In addition, drusen-related retinal disorders are often associated with adverse effects on the normal functioning of the retinal vasculature, which can impair nutrient and waste exchange, as well as cellular metabolism. Long-term consequences of such retinal disorders may include retinal degeneration, particularly of the macula, which can result in progressive and irreversible central vision loss.

Drusen-related retinal disorders are often genetically driven, in which one or more specific gene mutations have been identified, or wherein the gene or genes remain unidentified but there is nevertheless clear genetic inheritance. Still other forms appear not to have a strong genetic driver and may be idiopathic. Retinal disorders associated with drusen may often result in severe retinal damage and degeneration of the Retinal Pigment Epithelium (RPE). Some forms of drusen-related retinal disorders, such as Familial Drusen (FD), have a dominant inheritance and relatively early onset of pathology resulting in vision impairment that can occur as early as the fourth or fifth decade of life. Other forms of drusen-related retinal disorders have later-onset, such as age-related macular degeneration (AMD), which may show weaker genetic predispositions, and may have epigenetic influences. Regardless, in their most severe forms, drusen-related retinal disorders may result in degeneration of the RPE and development of geographic atrophy, which can progress to legal blindness.

There are no FDA-approved interventions to stop, reverse, prevent, or cure drusen-related retinal disorders, or to restore vision lost from such disorders. Thus, drusen-related retinal disorders present large unmet medical needs.

What is needed, then, is one or more treatments using therapeutic compositions and effective delivery methods for the treatment of drusen-related retinal disorders. The embodiments disclosed herein present a potential therapeutic for drusen-related retinal disorders.

### BRIEF SUMMARY

The present disclosure provides an improvement over other treatments for drusen-related retinal disorders, at least in part by introducing a novel method for treating such retinal disorders in a subject in need thereof. The present disclosure relates to a method that may comprise administering to the subject an effective amount of culture-expanded allogeneic mesenchymal stem cells to the subject's suprachoroidal area of the eye.

Mesenchymal stem cells are multipotent cells, and may sometimes be referred to as “mesenchymal stromal cells”, “medicinal signaling cells”, or “MSCs”. The term allogeneic may refer to cells that are not sourced from the patient to be treated.

In some aspects, the method may comprise a solution comprising the culture-expanded allogeneic mesenchymal stem cells and a balanced salt solution. Further, the solution of culture-expanded allogeneic mesenchymal stem cells and balanced salt solution may be free of human serum albumin. Additionally, the solution of culture-expanded allogeneic mesenchymal stem cells and balanced salt solution may be free of anti-coagulant excipients. In some as aspects, the solution of culture-expanded allogeneic mesenchymal stem cells may be free of heparin, which may include anti-clumping characteristics.

In some aspects, the method may comprise a solution comprising the culture-expanded allogeneic mesenchymal stem cells and a balanced salt solution containing less than 10% dimethyl sulfoxide. Additionally, the solution of culture-expanded allogeneic mesenchymal stem cells and balanced salt solution contains less than 1% dimethyl sulfoxide. In some aspects, the culture-expanded allogeneic mesenchymal stem cells and balanced salt solution may be free of dimethyl sulfoxide.

In some aspects, the methods described herein may include administration of an effective amount of allogeneic mesenchymal stem cells comprising an injection to the subject's suprachoroidal area of the eye.

In some aspects, the methods described herein may include administration of the effective amount of culture-expanded allogeneic mesenchymal stem cells that are effective to reduce geographic atrophy. Geographic atrophy can be analyzed by various methods that include fundus imaging, including, for example, fundus autofluorescence (FAF), microperimetry, optical coherence tomography (OCT), and color fundus photography or imaging, or other methods.

In some aspects, the reduction of geographic atrophy is determined by fundus autofluorescence at least seven days post-administration. In some aspects, the reduction of geographic atrophy may be determined by optical coherence tomography at least seven days post-administration. In some aspects, the reduction of geographic atrophy may be determined by color fundus photography or imaging at least seven days post-administration. In some aspects, the post-administration test results, or assessment, may be compared to pre-administration test results, or assessment.

In some aspects, the methods described herein may include administration of the effective amount of culture-expanded allogeneic mesenchymal stem cells that is effective at improving best-corrected visual acuity (BCVA), as assessed by various methods that may include the Early Treatment Diabetic Retinopathy Study (ETDRS) acuity test, the Minnesota Low-Vision Reading Test (MNREAD) Acuity Chart test, low-light visual acuity testing, or other methods, when comparing a pre-administration test result(s) to a post-administration test result(s). In some aspects, said post-administration test result(s) is at least seven days post-administration. Additionally, the methods described herein may include administration of the effective amount of culture-expanded allogeneic mesenchymal stem cells that are effective at reducing the decline in best-corrected visual acuity.

In some aspects, the methods described herein may include administration of the effective amount of culture-expanded allogeneic mesenchymal stem cells that is effective to improve the subject's best-corrected visual acuity (BCVA) score as assessed by the Early Treatment Diabetic Retinopathy Study (ETDRS) acuity test when comparing a pre-administration test result(s) to a post-administration test result(s), wherein said post-administration score is at least seven days post-administration.

In some aspects, the methods described herein may include administration of the effective amount of culture-expanded allogeneic mesenchymal stem cells that is effective to improve the subject's visual acuity may be assessed using the Minnesota Low-Vision Reading Test (MNREAD) Acuity Chart test when comparing a pre-administration test result(s) to a post-administration test result(s). In some aspects, said post-administration test result(s) is at least seven days post-administration.

In some aspects, the methods described herein may include administration of the effective amount of culture-expanded allogeneic mesenchymal stem cells that is effective to improve the subject's retina function as determined by microperimetry, including Macular Integrity Assessment (MAIA) microperimetry, when comparing a pre-administration assessment to a post-administration assessment. In some aspects, said post-administration assessment is at least seven days post-administration. Additionally, retinal function may be assessed by other microperimetry assessments when comparing a pre-administration assessment to a post-administration assessment. In some aspects, said post-administration assessment is at least seven days post-administration.

In some aspects, the methods described herein may include administration of an effective amount of culture-expanded allogeneic mesenchymal stem cells to improve one or more vision biomarker associated with the subject.

In some aspects, the one or more vision biomarker is selected from the group consisting of fundus imaging, geographic atrophy, drusen accumulation, optical coherence tomography, microperimetry, flicker sensitivity testing, or combinations thereof.

In some aspects, the methods described herein may include the effective amount of culture-expanded allogeneic mesenchymal stem cells administered to the subject include at least 5,000 cells.

In some aspects, the methods described herein may include the effective amount of culture-expanded allogeneic mesenchymal stem cells administered to the subject include at least 50,000 cells.

In some aspects, the methods described herein may include the effective amount of culture-expanded allogeneic mesenchymal stem cells administered to the subject include at least 5,000,000 cells. In one aspect, the methods described herein may include the effective amount of culture-expanded allogeneic mesenchymal stem cells administer to the subject is from 5,000 to 5,000,000 cells.

In some aspects, the effective amount of the culture-expanded allogeneic mesenchymal stem cells administered to the subject via suprachoroidal injection may be in a volume of 5 microliters to 500 microliters.

In some aspects, the methods disclosed herein may include administering culture-expanded allogeneic mesenchymal stem cells which are free of known genetic abnormalities underlying one or more retinal dystrophy characterized by drusen accumulation.

In some aspects, the methods disclosed herein may include administering culture-expanded allogeneic mesenchymal stem cells being delivered by a single or multiple suprachoroidal injections.

In some aspects, the methods described herein relate to a method for reducing geographic atrophy in a retina of a subject. In some aspects, the method may comprise providing an amount of solution including culture-expanded allogeneic mesenchymal stem cells and a balanced salt solution effective to reduce geographic atrophy in the retina.

In some aspects, the methods described herein may include injecting the solution into a suprachoroidal area of the subject.

In some aspects, the methods described herein may include measuring geographic atrophy prior to injecting the solution into the suprachoroidal area of the subject.

In some aspects, the methods described herein may include measuring geographic atrophy after injecting the solution into the suprachoroidal area of the subject. In some aspects, the measurement of geographic atrophy after injection may occur at least seven days post-injection.

In some aspects, the methods described herein may include the solution having culture-expanded allogeneic mesenchymal stem cells ranging from 5,000 cells to 5,000,000 cells.

In some aspects, the methods described herein may include measuring geographic atrophy of the subject includes imaging via fundus autofluorescence.

In some aspects, the methods described herein may include determining whether the subject suffers from drusen-related retinal disorder that is associated with familial drusen, or other inherited disorder.

In some aspects, the methods disclosed herein may include determining a drusen-related retinal disorder is caused, at least in part, by a genetic mutation.

In some aspects, the methods disclosed herein may include the genetic mutation resulting in familial drusen.

In some aspects, the methods disclosed herein may include the genetic mutation being associated with a gene for EFEMP1, CFI, or CFH.

In some aspects, the methods disclosed herein may include determining the drusen-related retinal disorder may be associated with a pattern of inheritance.

In some aspects, the methods disclosed herein may include determining the drusen-related retinal disorder is idiopathic.

In some aspects, the methods disclosed herein may include administering culture-expanded allogeneic mesenchymal stem cells being sourced from umbilical cord, bone marrow, adipose tissue, Wharton's jelly, placenta, pluripotent stem cells, or induced pluripotent stem cells.

In some aspects, the methods disclosed herein may include administering an effective amount of the culture-expanded allogeneic mesenchymal stem cells for reducing or inhibiting a rate of retinal degeneration. In some aspects, the methods disclosed herein may include administering an effective amount of the culture-expanded allogeneic mesenchymal stem cells for inhibiting retinal degeneration progression.

### BRIEF DESCRIPTION OF THE SEVERAL VIEWS OF THE DRAWINGS

FIG. 1 is a comparison of MAIA microperimetry data showing baseline data pre-treatment and data at 7 days post-treatment.

FIG. 2 is a comparison of MAIA microperimetry data showing baseline data pre-treatment and data at 28 days post-treatment.

FIG. 3 is a comparison of FAF showing GA baseline data pre-treatment and GA data at 7 days post-treatment.

FIG. 4 is a comparison chart of BCVA scores via the Early Treatment Diabetic Retinopathy Study (ETDRS) acuity test at Day 0, Day 7 and Day 28.

FIG. 5 is a comparison chart of Minnesota Low-Vision Reading Test (MNREAD) Acuity Chart test scores at Day 0 and Day 28.

FIG. 6 is an embodiment of a treatment method schedule for treatment days and follow-up days.

FIG. 7 is an embodiment of a treatment schedule for pre-treatment, treatment days, and post-treatment.

### DETAILED DESCRIPTION

Familial Drusen, which may also be referred to herein as dominant drusen, is a retinal dystrophy and, more specifically, a drusen-related retinal disorder, which may show an inheritance pattern that is often dominant, and may often lead to legal blindness in the forties to fifties year of age. Familial Drusen may be generally categorized into four classes. One includes a 1033 C>T mutation of the EGT-containing fibulin-like extracellular matrix protein 1 gene (EFEMP1), which results in a R345W codon change. This mutation leads to a specific Familial Drusen also known as Doyne honeycomb retinal dystrophy or Malattia Leventinese. A second class includes mutations of the complement factor H gene (CFH), which has several missense and splice variant mutations. A third class consists of mutations of the complement factor I gene (CFI; variants of which may also be associated with AMD). And a fourth class is of unknown origin, not entailing one of the above 3 genes, but yet has a demonstrated pattern of inheritance. Other classes of Familial Drusen arguably also exist, such as: Sorsby fundus dystrophy (SFD), which may be caused by mutations in the tissue inhibitor of metalloproteinases-3 gene (TIMP3); Sorsby Pseudoinflammatory Fundus Dystrophy; North Carolina Macular Dystrophy, which entails mutation in the MCDR1 gene; and others.

One can also identify an inflammatory component and drusen accumulation in each of these conditions that may be the result of, or at least due in part to, impairment of the vasculature to remove waste products. In addition to other mechanisms of actions disclosed herein, the use of allogeneic mesenchymal stem cells (MSC) as a treatment for Familial Drusen and other drusen-related retinal disorders may benefit patients by inducing regenerative responses, reducing inflammation and fibrosis, and improving vascular function for nutrient/waste management. These activities may result from mesenchymal stem cell secretion of proteins and exosomes, direct cell-cell interactions, and even via direct organelle exchange through nanotube bridges. The mechanisms of action are not limiting, but only exemplary of the various aspects disclosed herein.

While Familial Drusen may be one disorder that is treated with the treatment methods, compounds, and formulations, and other disclosed subject matter in the various aspects of this specification, other drusen-related retinal disorders may also be treated by the same. Drusen-related retinal disorders may be characterized by pathological accumulation of extracellular drusen deposits in the retina, and concomitant chronic inflammation of the retina, impairment of retina pigment epithelial cell function, and geographic atrophy. In addition, drusen-related retinal disorders are associated with adverse effects on the normal functioning of the retinal vasculature that can impair nutrient and waste exchange, as well as impaired cellular metabolism. Long-term consequences lead to retinal degeneration, particularly of the macula, which can result in permanent and irreversible vision loss, and particularly central vision loss. Drusen-related retinal disorders are generally predicated on the deposition and accumulation of drusen deposits. Genetic factors can lead to the development of certain drusen-related retinal disorders, some of which specific genes and gene mutations have been identified, and others of which the affected genes and/or mutations remain unknown. Other forms of drusen-related retinal disorders may not have a strong or weak genetic influence, and may be idiopathic. While genetic forms of drusen-related retinal disorder often have an earlier onset, and often result in retinal damage and/or legal blindness before the age of sixty years, drusen-related retinal disorders without a strong genetic component may not result in detrimental effects on the retina and vision until after the age of sixty years. Nevertheless, drusen-related retinal disorders with later onset may be influenced by genetic factors.

While there may be different forms of drusen-related retinal disorders, the overall shared pathology, disease progression, and symptoms remain similar between them, such that one may appreciate the subject matter disclosed in this specification is relevant to drusen-related retinal disorders generally.

Mesenchymal stem cells are multipotent cells that have been recognized as having pro-regenerative properties. Because of the beneficial properties, they may provide promising treatment options for treating drusen-related retinal disorders. Mesenchymal stem cells, while not being limited to the following, may act with anti-inflammatory, immunomodulatory, anti-fibrotic, pro-vascular, and/or pro-regenerative processes. Thus, mesenchymal stem cells may be understood to have the potential to target and treat multiple pathological targets implicated in retinal disorders, including drusen-related retinal disorders, some of which have inherited forms.

Mesenchymal stem cells are found in, and may be isolated from, various tissue, such as umbilical cord, bone marrow, adipose tissue, Wharton's jelly, dental pulp, and placenta, and can be culture-expanded ex vivo to high homogeneity.

Mesenchymal stem cells exhibit immunoprivileged or immunoevasive properties, due at least in part to undetectable surface-expression of major histocompatibility complex type II (MHC-II) and low levels of major histocompatibility complex type I (MHC-I) surface-expression. These properties, at least in part, allow for mesenchymal stem cells to be used for allogeneic treatments that may not require tissue-type matching. Certain aspects, disclosed herein, may use culture-expanded allogeneic mesenchymal stem cells as part of the treatment regimen for drusen-related retinal disorders, including, but not limited to, Familial Drusen.

In aspects as disclosed herein, culture-expanded allogeneic mesenchymal stem cells may be used to slow, disrupt, halt, and/or even reverse the progressive damage to the retina caused by drusen-related retinal disorders, as well as the associated effects on other ocular areas. Treatments may include, but are not limited to, treating progressive geographic atrophy, progressive drusen accumulation, associated chronic inflammation, and/or progressive loss of vision, including central and mid-peripheral visual field.

In certain aspects disclosed herein, umbilical cord-derived allogeneic mesenchymal stem cells may be used to treat drusen-related retinal disorders, including those as a result of genetic inheritance and/or predisposition. In some aspects, the method of treatment may include suspension of culture-expanded allogeneic mesenchymal stem cells in a balanced salt solution. In some aspects, delivery of the allogeneic mesenchymal stem cells is into the suprachoroidal space of a patient in need of said treatment for retinal disorders, including for drusen-related retinal disorders. In some aspects, the route of administration for treatment may include a single injection or multiple injections per treatment. In some aspects, the multiple injection sites may be used when multiple injections are administered. Some aspects may include treatments that can be performed multiple times over weeks, months, and/or years. The use of culture-expanded allogeneic mesenchymal stem cells may treat drusen-related retinal disorders, regardless of underlying etiology.

Certain gene variant(s) and/or gene mutation(s) associated with the drusen-related retinal disorders may be known, including, but are not limited to, the EFEMP1, CFI, and CFH genes, and other genes, as well as unknown gene variants. Embodiments disclosed herein may treat all currently known drusen-related retinal disorders and retinal dystrophies regardless of genotype and/or phenotype as related to the disease.

It may also be appreciated that the disorders may be coded under any of the following ICD-11 codes or comparable code(s) as the ICD may be updated: 9B70—Inherited retinal retinopathies; 9B71—Retinopathy; 9B72—Inflammatory diseases of the retina; 9B75—Macular disorders; 9B78—Certain specified retinal disorders; 9B7Y—Other specified disorders of the retina; 9B7Z—Disorders of the retina, unspecified.

In some aspects, the disorder as presented by the patient may or may not have accompanying geographic atrophy (GA).

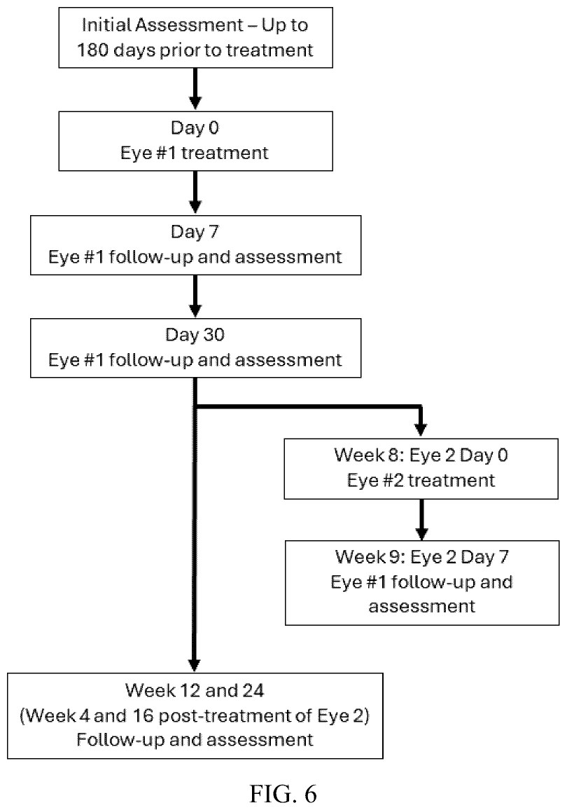

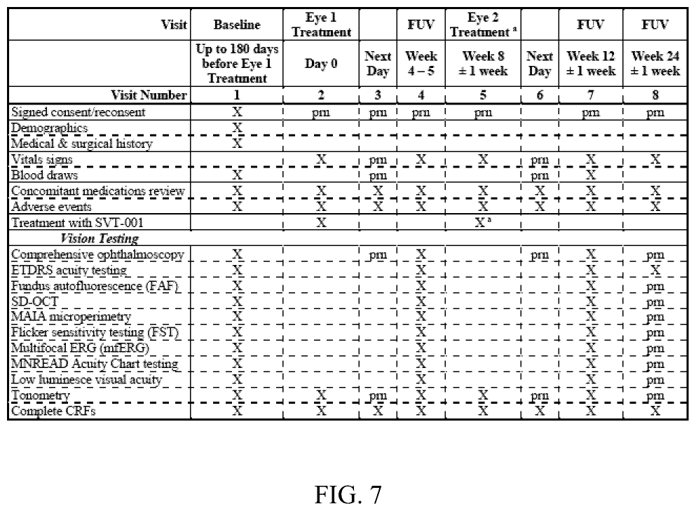

The treatment compositions and methods disclosed herein may be performed on a subject in need thereof, wherein one or both eyes of the subject are treated. As shown in FIGS. 6 and 7, in one aspect, a subject may undergo a treatment regimen wherein an initial assessment of the subject is performed. The initial assessment may assess various physiological, genetic, and molecular markers, phenotypes, symptoms, and the like, to determine the candidacy of the subject for treatment by the methods disclosed herein. The initial assessment may be conducted at any time prior to the day of treatment, including up to 180 days pre-treatment, or 1 year pre-treatment, or up to 5 years pre-treatment. In some aspects, a single eye may be treated (Day 0). In some aspect, both eyes may be treated. In aspects in which both eyes are treated, the treatment for each eye may occur simultaneously, such that both eyes are treated on the same day. In other aspects in which both eyes are treated, the treatment for each eye may occur consecutively such that there is an offset of treatment days for the subject's first eye and the subject's second eye. In some aspects, the offset may be on the order of days, weeks, months, or years. As one exemplary aspect, depicted in FIG. 6, a subject may receive a first treatment to eye #1 and then receive a first treatment to eye #2 approximately 8 weeks later.

As shown in FIGS. 6 and 7, a subject may undergo follow-up assessments after treatment of one or both of the subject's eyes. In one aspect, the subject may undergo follow-up assessments on Day 7, Day 30, week 12, and week 24. These follow-up assessments dates are not limiting, but only one example of a schedule which may be implemented by the treatment methods disclosed herein. When a subject receives a first treatment for a first eye, and a second treatment for the second eye, the follow-up schedule of the second eye may be the same as the follow-up schedule of the first eye, but be off-set by the date of treatment from the first eye to the second eye.

The treatment compositions and methods disclosed herein may lead to reduced retinal dysfunction and damage, or improvements therein, as assessed via one or more biomarkers, including, but not limited to the following: maintain or improve fundus imaging, such as fundus autofluorescence (FAF) imaging, slow down pathology thereof, decrease and/or stop and/or reverse geographic atrophy (GA), decrease and/or inhibit a rate of and/or reverse retinal degeneration, maintain or improve fundus appearance, or slow down pathology thereof. In some aspects, the treatment compositions and methods disclosed herein may stop further drusen accumulation and/or decrease drusen size and load or slow down pathology thereof. In some aspects, the administration of treatment compositions may inhibit or stop the retinal degeneration progression, and in some aspects may reverse the retinal degeneration progression. In some aspects, the treatment compositions and methods disclosed herein may maintain or improve retinal cytoarchitecture as assessed by optical coherence tomography (OCT), such as spectral-domain OCT (SD-OCT), or slow down pathology thereof. In some aspects disclosed herein, the treatment compositions and methods may maintain or improve microperimetry, or slow down pathology thereof, including as assessed by macular integrity assessment (MAIA) microperimetry. In some aspects, the treatment compositions and methods disclosed herein may maintain or improve electroretinography (ERG), or slow down pathology thereof, including as assessed by multifocal ERG (mfERG). In some aspects, the treatment compositions and methods disclosed herein may maintain or improve flicker sensitivity testing (FST) or slow down pathology thereof.

In some aspects, the treatment may promote improved visual acuity, or slow down the decline thereof, as assessed via any of the following: Early Treatment Diabetic Retinopathy Study (ETDRS) acuity testing; the Minnesota Low Vision Reading Test (MNREAD) Acuity Chart testing; Low-luminescence visual acuity testing.

Prior to treatment, as well as post-treatment, in some aspects, various vision testing, including the foregoing tests previously discussed, may be conducted to assess a patient's visual acuity as well as overall ocular health and pathology. In some aspects, the following tests/assessments may be conducted, but this is not meant to be a limited listing: comprehensive ophthalmoscopy, early treatment diabetic retinopathy study (ETDRS) acuity testing, fundus imaging, fundus autofluorescence (FAF), optical coherence tomography (OCT), spectral-domain optical coherence tomography (SD-OCT), microperimetry, macular integrity assessment (MAIA), flicker sensitivity testing (FST), Minnesota low vision reading test (MNREAD) acuity chart testing, low luminesce visual acuity, and/or tonometry, and combinations thereof.

It will also be appreciated that while many of the retinal disorders discussed herein are drusen-related, the embodiments disclosed herein may also be compositions and methods of use to treat other retinal disorders that are not drusen-related.

In some aspects, the compositions disclosed herein may include mesenchymal stem cells that are sourced from bone marrow. In certain aspects, the compositions may include mesenchymal stem cells that are sourced directly from other tissue(s) or tissue culture(s), such as umbilical cord, adipose tissue, Wharton's jelly, or induced pluripotent stem cells (iPSCs). These mesenchymal stem cells may be referred to herein as culture-expanded mesenchymal stem cells. In certain aspects, the compositions may include mesenchymal stem cells that may be derived from frozen stocks. In certain aspects, the compositions may include mesenchymal stem cells that may be suspended in xenogeneic-free medium. In certain aspects, the compositions may include mesenchymal stem cells that may be suspended in a solution which is free of human serum albumin (HSA). In some aspects, the compositions may include mesenchymal stem cells that may be suspended in a balanced salt solution which is free of other excipients that may normally be isolated from blood or serum, or normally be isolated from tissues other than the retina, suprachoroidal space, vitreous humor, and other intraocular structures exclusive of its vasculature. In some aspects, the compositions may include mesenchymal stem cells that may be suspended in a balanced salt solution which is also free of heparin and/or other anti-coagulant or pro-coagulant excipients. In certain aspects, the compositions may include mesenchymal stem cells that may be suspended in a balanced salt solution that is free of hetastarch as an excipient. In some aspects, the hetastarch may be Hespan®. In certain aspects, the balanced salt solution may be about 0.9% saline (sodium chlorite) or similar solution. It will be understood that a balanced salt solution may be a solution with a physiologically tolerable pH. In some aspects, the balanced salt solution may also include an isotonic salt concentration. In certain aspects, the balanced salt solution may be used to hydrate and/or protect tissue and cells. In certain aspects, the balanced salt solution may include dimethyl sulfoxide (DMSO). In some aspects, the solution may include less than 10% DMSO, may include less than 4% DMSO, may include less than 3% DMSO, may include less than 2% DMSO, may include less than 1% DMSO, or may include from 0.00001% to 1.0% DMSO. In some aspects, the balanced salt solution may include 0.0% DMSO. It should be understood that any recitation of “less than” may include some or none of the composition.

Preparations of allogeneic mesenchymal stem cells for other indications found in the prior art, particularly for intravenous infusion, are typically formulated with excipients using one or more of the above (i.e., human serum albumin, heparin, hetastarch, etc.). However, the introduction of such excipients in the eye could present serious adverse consequences. For example, the introduction of heparin or other anticoagulants into the eye, and especially in proximity to the choriocapillaris during suprachoroidal delivery, could potentially cause blood vessel leakage. Another example is the introduction of human serum albumin into the suprachoroidal area. Since human serum albumin is normally in the blood, introduction into the extravascular suprachoroidal space has potential to mimic blood vessel leakage, and in turn unintended and adverse injury-response reaction, or other consequences.

In certain aspects, the culture-expanded allogeneic mesenchymal stem cells do not contain deleterious mutations implicated in visual dysfunction, such as, but not limited to, the EFEMP1 1033C>T (R345W) or CFH1 196646753 (C192F) missense variants.

In some aspects, a treatment may include a total delivery of culture-expanded allogeneic mesenchymal stem cells at one or more of the following: about 5,000 cells; about 10,000 cells; about 15,000 cells; about 20,000 cells; about 25,000 cells; about 5,000 to 50,000 cells; about 25,000 to 100,000 cells; about 100,000 to 5,000,000 cells; and/or >5,000,000 cells. In certain aspects, the total delivery of allogeneic mesenchymal stem cells may be about 25,000 cells, may be about 30,000 cells, may be about 35,000 cells, may be about 40,000 cells, may be about 45,000 cells, may be about 50,000 cells, may be about 55,000 cells, may be about 60,000 cells, may be about 65,000 cells, may be about 70,000 cells, may be about 75,000 cells, may be about 80,000 cells, may be about 85,000 cells, may be about 90,000 cells, may be about 95,000 cells, may be about 100,000 cells, may be about 200,000 cells, may be about 300,000 cells, may be about 400,000 cells, and/or may be about 500,000 cells. Said treatment may be a single treatment or multiple treatments. In certain aspects, cells may be delivered in the range one of the following total volumes: <5 microliters; about 5-200 microliters; about 5-300 microliters; about 5-400 microliters; about 5-500 microliters; >200 microliters; and/or >500 microliters. In certain aspects, the concentration of cells may be in the range of one of the following: about 5,000 cells/mL; about 25,000 cells/mL; about 50,000 cells/mL; may be about 30,000 cells/mL, may be about 35,000 cells/mL, may be about 40,000 cells/mL, may be about 45,000 cells/mL, may be about 50,000 cells/mL, may be about 55,000 cells/mL, may be about 60,000 cells/mL, may be about 65,000 cells/mL, may be about 70,000 cells/mL, may be about 75,000 cells/mL, may be about 80,000 cells/mL, may be about 85,000 cells/mL, may be about 90,000 cells/mL, may be about 95,000 cells/mL, may be about 100,000 cells/mL, may be <100,000 cells/mL; may be about 100,000-5,000,000 cells/mL; may be about >5,000,000 cells/mL.

While not being limited to the following, certain mechanisms of action (MOA) may play a role in the treatment process. In some aspects, the mechanism of action of the allogeneic mesenchymal stem cells may include one or more of the following: anti-inflammatory, immuno-regulatory, anti-fibrotic, pro-vascular, and pro-regenerative. In certain aspects, the compositions and treatment disclosed herein may decrease retinal inflammation, decrease and/or cease further retinal degeneration, and/or promote improved functioning of the retinal pigment epithelium (RPE) and RPE cells. In some aspects, the compositions and treatments disclosed herein may result in improved vascular flow in the eye without promoting pathological neo-angiogenesis.

Supporting the disclosure of allogeneic mesenchymal stem cells for the treatment of retinal disorders, treatment of a patient with culture-expanded allogeneic mesenchymal stem cells was undertaken. The patient received a single treatment dose of culture-expanded allogeneic mesenchymal stem cells to a single eye. The therapeutic was administered via suprachoroidal injection in order to reduce serious risks associated with current standard of care routes of administration, e.g., injection directly into the vitreous humor or subretinal injection. The risks of current routes of administration can include retinal detachment, vitreous detachment, pain, scarring, increased intraocular pressure, and/or hemorrhage of blood vessels in the sclera. The treatment of the patient may be understood as Day 0, in which the patient received a single suprachoroidal injection. The patient's follow-up visits were Day 1 and Day 7, and weeks 4, 8, 12, and 24. It is expected that a simultaneous or consecutive treatment plan for a subject's second eye, if to be treated, would also be subject to a similar treatment plan and yield similar treatment results. FIG. 6 is illustrative of one embodiment in which a treatment protocol may be used, but such treatment options are not limited solely to FIG. 6.

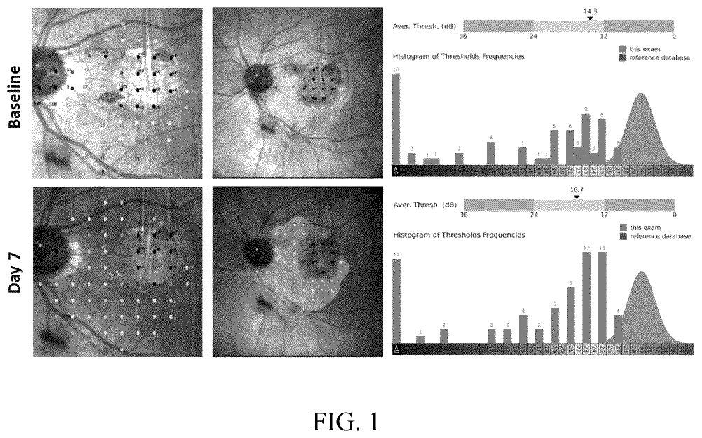

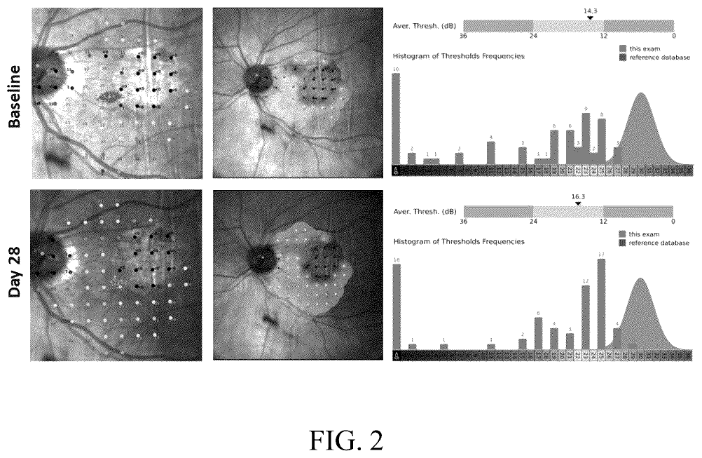

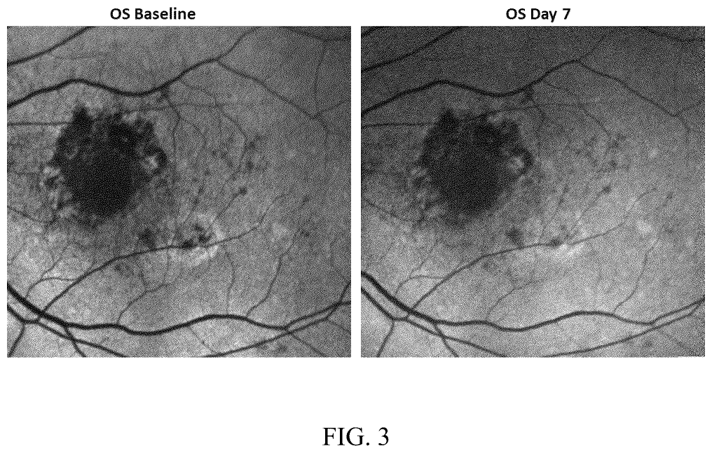

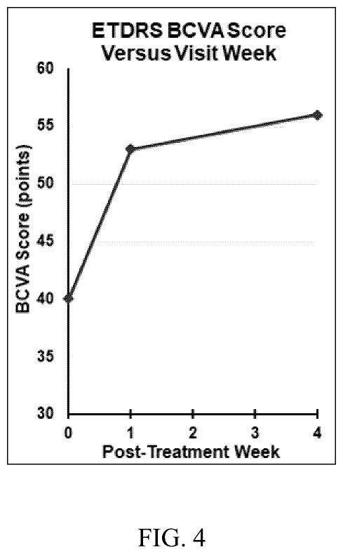

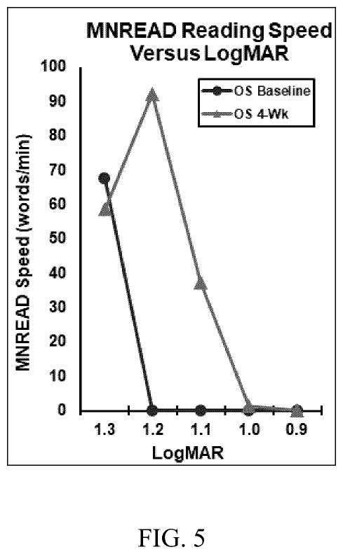

It has been observed that the treatment is well-tolerated by a patient with respect to both the therapeutic agent and the injection method. Improvements were observed across vision-related biomarkers within days of treatment, including, but not limited to, increased macular sensitivity and visual field, as evidenced by Macular Integrity Assessment (MAIA) microperimetry (FIGS. 1 and 2), fundus appearance (FIG. 3), improvement in Best Corrected Visual Acuity (BCVA) via the Early Treatment Diabetic Retinopathy Study (ETDRS) acuity test (FIG. 4), and improvement in the Minnesota Low-Vision Reading Test (MNREAD) Acuity Chart test (FIG. 5). Intraocular pressure remained stable. Various biomarkers were assessed both pre-administration of the culture-expanded allogeneic mesenchymal stem cells and post-administration. It may be appreciated that administration of an effective amount of the culture-expanded allogeneic mesenchymal stem cells improves one or more vision biomarkers associated with the subject. The vision biomarker may be selected from the group consisting of, but not limited to, fundus imaging, geographic atrophy, drusen accumulation, retinal cytoarchitecture as assessed by optical coherence tomography, microperimetry, flicker sensitivity testing, and combinations thereof.

As shown in FIG. 1, the Macular Integrity Assessment (MAIA) microperimetry revealed improvement of a patient within 1-week post-treatment in the allogeneic mesenchymal stem cell-treated eye (bottom panels—OS DAY 7) from pre-treatment (top panels—OS Baseline). The images provided on the left side and middle include the microperimetry results that are correlated with and superimposed on the patient's retina images. It should be noted that the portions of the left side images with black dots denoted by “<0” indicate non-responsive areas. Comparison of the superimposed images of the pre-treatment versus post-treatment with allogeneic mesenchymal stem cells revealed that the number of responsive areas increased, as well as magnitude of sensitivities (higher numbers indicate increased response). The data and graphs provided on the right side are graphical representations of the improvement showing a post-treatment right shift towards more normal response. FIG. 2 shows MAIA microperimetry comparisons of Day 0 versus Day 28 (4 weeks) for the treated eye.

In FIG. 3, the images were produced by Fundus autofluorescence (FAF) and indicate a reduction in geographic atrophy (GA) by seven days post-treatment. FAF is a current standard imaging technology for assessing geographic atrophy. The left image is pre-treatment (OS Baseline) and when compared to the image on the right (OS Day 7), geographic atrophy appeared reduced at Day 7.

In FIG. 4, the Early Treatment Diabetic Retinopathy Study (ETDRS) acuity test using Best-Corrected Visual Acuity (BCVA) showed an improvement in the patient from 40 (baseline: 1 day prior to treatment) to 53 by seven days post-treatment, and 56 by 28 days post-treatment (FIG. 4). It will be appreciated that this equates to an improvement of visual acuity on the Snellen chart from approximately 20/160 to better than 20/80 at 28 days post-treatment.

In FIG. 5, Minnesota Low-Vision Reading Test (MNREAD) Acuity Chart test showed both improvements in reading speed and visual acuity at 4 weeks post-treatment.

In assessing an individual's progression after treatment as disclosed herein, one or more baseline measurements may be taken to establish pre-treatment values and statuses for comparison to post-treatment measurements. In some aspects, the baseline measurements may be taken within 180 days prior to treatment. The baseline measurements may be performed over multiple days. In some aspects, the data/measurements/value that are acquired prior to treatment, from 180 days pre-treatment up to day 0 of treatment, may be used for the individual's baseline measurements. In some aspects, the baseline measurements may be taken within 1-5 years prior to treatment.

The following listing of assessments and/or tests disclose the general methodology for performing said assessments and/or tests.

Comprehensive ophthalmoscopy: Standard comprehensive ophthalmoscopy may be performed to examine external and internal eye structures and may include slit-lamp examination.

Early Treatment Diabetic Retinopathy Study (ETDRS) acuity testing: ETDRS acuity testing is a validated method for measuring visual acuity and considered a gold standard for testing visual acuity. It uses an illuminated standardized letter chart at which the test subject stands 4 meters away, and in which each consecutive row of letters corresponds to a specific visual acuity Snellen fraction measure (e.g., 20/20, etc.). Logarithm scoring of the minimum angle of resolution (log MAR) units may be used.

Fundus autofluorescence (FAF): FAF is a non-invasive imaging technique that may be used to assess geographic atrophy (GA) and other retinal structural changes. GA may be understood to be the loss of RPE cells and photoreceptors in the macula, leading to central vision loss. GA may be clearly delineated as hypo-autofluorescent (dark) regions on FAF images.

Spectral-domain optical coherence tomography (SD-OCT): SD-OCT is an advanced high-resolution imaging technique that uses near-infrared light (800-900 nm) to visualize cross-sectional images of eye structures, including the retinal microstructures (e.g., retinal cells, blood vessels, and RPE), choroid, and optic nerve. SD-OCT may be used to quantitatively analyze changes in segmented retinal layers, including ellipsoid zone (EZ), i.e., the photoreceptor inner segment-outer segment junction and inner segment-outer segment (IS/OS) junction.

MAIA Microperimetry: MAIA Microperimetry combines fundus imaging with perimetry to allow precise correlation of retina function response and anatomical structures. Key features may include light sensitivity mapping, high resolution imaging, eye tracking, and fixation stability analysis.

Flicker sensitivity testing (FST): FST may be used to assess integrity of rod and cone-mediated vision via the subject's ability to detect rapid light flickers directed to the retina. Cone-mediated FST may be used to evaluate photopic vision and uses brighter intensities at varying wavelengths under bright lighting conditions. Rod-mediated FST may be used to evaluate scotopic vision using low intensity flickers and longer light wavelength under dim lighting conditions.

Minnesota Low-Vision Reading Test (MNREAD) Acuity Chart test: This highly sensitive visual acuity test may be used to evaluate reading performance, including reading speed and acuity, using reading charts of varying sizes and contrasts. It is commonly used in clinical trials to evaluate patients with impaired vision.

Low luminesce visual acuity: As the name implies, this test may be used to evaluate visual acuity under low-light conditions.

Tonometry: Standard tonometry may be used to examine internal ocular pressure (IOP).

FIG. 7 is an embodiment of a treatment method schedule for treatment days and follow-up days.

FIG. 7 is an embodiment of a treatment schedule for pre-treatment, treatment days, and post-treatment.

Thus, although there have been described particular embodiments of the present invention of a new and useful allogeneic mesenchymal stem cells for the treatment of retinal disorders associated with the presence of drusen it is not intended that such references be construed as limitations upon the scope of this invention except as set forth in the following claims.

Thus, although there have been described particular embodiments of the present invention of a new and useful ALLOGENEIC MESENCHYMAL STEM CELLS FOR THE TREATMENT OF RETINAL DISORDERS ASSOCIATED WITH THE PRESENCE OF DRUSEN it is not intended that such references be construed as limitations upon the scope of this invention except as set forth in the following claims.

What is claimed is:

1. A method of treating drusen-related retinal disorders in an eye of a subject in need thereof, wherein the method comprises administering to the subject an effective amount of culture-expanded allogeneic mesenchymal stem cells to a suprachoroidal area of the subject's eye.

2. The method of claim 1, further comprising determining the drusen-related retinal disorder is caused, at least in part, by a genetic mutation.

3. The method of claim 2, wherein the genetic mutation results in familial drusen.

4. The method of claim 2, wherein the genetic mutation is associated with a gene for EFEMP1, CFI, or CFH.

5. The method of claim 1, further comprising determining the drusen-related retinal disorder is associated with a pattern of inheritance.

6. The method of claim 1, wherein the drusen-related retinal disorders is idiopathic.

7. The method of claim 1, wherein the method comprises a solution containing the culture-expanded allogeneic mesenchymal stem cells and a balanced salt solution.

8. The method of claim 7, wherein the solution does not contain one or more excipients of human serum albumin, anti-coagulant, heparin, or hetastarch.

9. The method of claim 7, wherein the solution further comprises less than 10% dimethyl sulfoxide.

10. The method of claim 1, wherein administration of the effective amount of the culture-expanded allogeneic mesenchymal stem cells comprises an injection to the subject's suprachoroidal area of the eye.

11. The method of claim 1, wherein administration of the effective amount of the culture-expanded allogeneic mesenchymal stem cells is effective to reduce geographic atrophy, as assessed by fundus imaging, optical coherence tomography, microperimetry, or the like, when comparing a pre-administration to a post-administration assessment, wherein said post-administration assessment is at least seven days post-administration.

12. The method of claim 1, wherein administration of the effective amount of the culture-expanded allogeneic mesenchymal stem cells improves a best-corrected visual acuity of the subject as assessed by ETDRS, low-light visual acuity, or Minnesota Low-Vision Reading Test (MNREAD) Acuity Chart test, or the like, when comparing a pre-administration to a post-administration assessment, wherein said post-administration assessment is at least seven days post-administration.

13. The method of claim 1, wherein administration of the effective amount of the culture-expanded allogeneic mesenchymal stem cells is effective to improve visual field testing in the subject, including as assessed by microperimetry or MAIA microperimetry, when comparing a pre-administration assessment to a post-administration assessment, wherein said post-administration assessment is at least seven days post-administration.

14. The method of claim 1, wherein administration of the effective amount of the culture-expanded allogeneic mesenchymal stem cells improves a vision biomarker associated with the subject.

15. The method of claim 14, wherein the vision biomarker is selected from the group consisting of fundus imaging, geographic atrophy, drusen accumulation, optical coherence tomography, microperimetry, flicker sensitivity testing, or combinations thereof.

16. The method of claim 1, wherein the effective amount of the culture-expanded allogeneic mesenchymal stem cells is 5,000 to 5,000,000 cells.

17. The method of claim 1, wherein the effective amount of the culture-expanded allogeneic mesenchymal stem cells administered to the subject via suprachoroidal injection is in a volume of 5 microliters to 500 microliters.

18. The method of claim 1, wherein the culture-expanded allogeneic mesenchymal stem cells are free of known genetic abnormalities underlying one or more retinal dystrophy characterized by drusen accumulation.

19. The method of claim 1, wherein the culture-expanded allogeneic mesenchymal stem cells are delivered by a single or multiple suprachoroidal injections.

20. The method of claim 1, wherein the drusen-related retinal disorder includes geographic atrophy of the subject's eye, and wherein the method further comprises measuring the geographic atrophy prior to injection of the culture-expanded allogeneic mesenchymal stem cells into the suprachoroidal area of the subject's eye, and at least seven days after injection of the culture-expanded allogeneic mesenchymal stem cells into the suprachoroidal area of the subject's eye.

21. The method of claim 1, wherein the method further comprises determining the drusen-related retinal disorder includes geographic atrophy being associated with familial drusen, or other inherited disorder.

22. The method of claim 1, wherein the method further comprises the allogeneic mesenchymal stem cells being sourced from umbilical cord, bone marrow, adipose tissue, Wharton's jelly, placenta, pluripotent stem cells, induced pluripotent stem cells, or other human source.

23. The method of claim 1, wherein administration of the effective amount of the culture-expanded allogeneic mesenchymal stem cells reduces or inhibits a rate of retinal degeneration.

24. The method of claim 1, wherein administration of the effective amount of the culture-expanded allogeneic mesenchymal stem cells inhibits retinal degeneration progression.
